(19) United States
(12) Patent Application Publication
(10) Pub. No.: US 2024/0342242 A1
(43) Pub. Date: Oct. 17, 2024

(54) COMPOSITIONS AND METHODS FOR TREATING MASTITIS

(71) Applicant: Zoetis Services LLC, Parsippany, NJ (US)

(72) Inventors: Hilary Phelps, Jackson, MI (US); Brianna Joy Pomeroy Beam, Kalamazoo, MI (US); Richard Andrew Ewin, Kalamazoo, MI (US); Christopher A. Zook, Kalamazoo, MI (US); Michael Kuhn, Kalamazoo, MI (US); Xi Zeng, Kalamazoo, MI (US)

(21) Appl. No.: 18/294,706

(22) PCT Filed: Aug. 5, 2022
(86) PCT No.: PCT/US2022/074570

§ 371 Date: Feb. 2, 2024

Related U.S. Application Data
(60) Provisional application No. 63/229,699, filed on Aug. 5, 2021.

(51) Int. Cl. A61K 38/17; A61K 9/00; A61P 15/14; A61P 31/04; A61P 37/04

(52) U.S. Cl. CPC A61K 38/1729; A61K 9/0041; A61P 15/14; A61P 31/04; A61P 37/04

(57) ABSTRACT

The invention provides compositions and methods for treating mastitis. Specifically, the invention provides cathelicidin peptides for treating mastitis.

### CROSS-REFERENCE TO RELATED APPLICATIONS

This application claims priority to and the benefit of U.S. Provisional Patent Application 63/229,699, filed Aug. 5, 2021, which is incorporated by reference herein in its entirety.

### FIELD OF THE INVENTION

The invention relates to compositions and methods for treating mastitis. Specifically, the invention relates to cathelicidin peptides for treating mastitis.

### BACKGROUND OF THE INVENTION

Mastitis has important deleterious effects on dairy herd productivity, longevity, and profitability due to decreased milk production, decreased reproductive performance, costs associated with treatments, and increased risk of culling and death of affected animals.

Mastitis has been reported to be the most important cause of antimicrobial drug use in dairy herds with about half of the antimicrobials used on dairies for mastitis control.

Treatment of mastitis is given on the premise that costs will be outweighed by production gains resulting from elimination of infection. Most farms have established mastitis management programs and include strategies such as routine whole herd antibiotic therapy, culling of chronically affected cows, post-milking teat disinfection, as well as ensuring routine maintenance of milking machines. Due to high treatment costs, lost income due to discarded milk, public health, and animal welfare concerns, it would be advantageous for dairy cattle to resist or mount effective immune responses to clear the wide variety of mastitis-causing pathogens.

Despite the fact that infectious disease remains a significant threat to food security and animal welfare, broad antimicrobial treatment and prevention practices in the dairy industry are under ever increasing pressure from regulatory authorities as well as consumers of dairy products. Non-antibacterial immunomodulators can serve to fill this important void.

Current immunoprophylactic strategies with vaccines against mastitis pathogens only show very limited effects against natural intramammary infection (IMI).

Accordingly, there exists a need for a non-antibacterial immunomodulator for treating mastitis.

### SUMMARY OF THE INVENTION

In one aspect, the invention provides a method for treating mastitis in a subject, the method comprising: administering to said subject an effective amount of cathelicidin 2 (CATH2) peptide or a variant thereof, thereby treating said mastitis in said subject. In an exemplary embodiment, the peptide of the invention is an immune modulatory peptide having no antibiotic activity. In some embodiments, the peptide's intrinsic antimicrobial activity is abrogated with milk or milk proteins.

In another aspect, the invention provides a composition comprising: cathelicidin 2 (CATH2) peptide or a variant thereof. In an exemplary embodiment, the composition is an intra-mammary delivery composition.

In yet another aspect, the invention provides a device comprising: a chamber for storing a composition, wherein said composition comprises cathelicidin 2 (CATH2) peptide or a variant thereof. In an exemplary embodiment, the device is an intra-mammary delivery device.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows that CATH2 peptide regulates cytokine production following stimulation with TLR 4 agonists.

FIG. 2 shows CATH2 antibacterial activity inhibition in the presence of milk.

FIG. 3 shows clinical mastitis incidence of an E. coli mastitis model.

FIG. 4 shows bacterial load in the milk by time point and study day in an E. coli clinical mastitis model.

FIG. 5 shows rectal temperatures by timepoint and study day in an E. coli clinical mastitis model.

FIG. 6 shows somatic cell count by timepoint and study day in an E. coli clinical mastitis model.

FIG. 7 shows milk Yield as measured by AfiLab in line system at each milking for the study period in an E. coli clinical mastitis model.

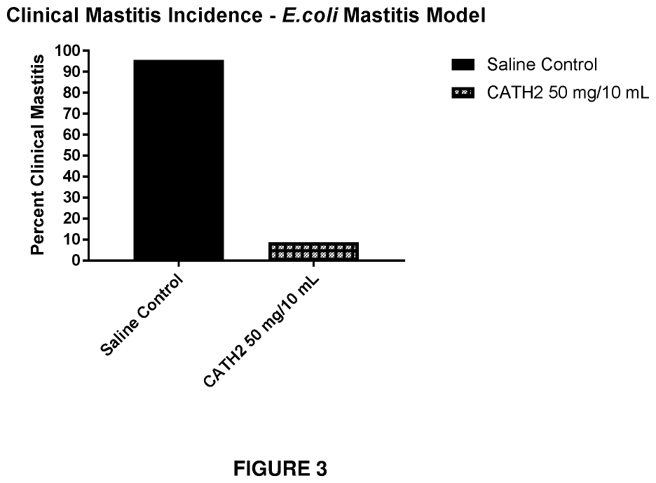

FIG. 3 shows bovine serum albumin (BSA) in the milk by time point and study day in an E. coli clinical mastitis model.

FIG. 9 shows the percentage of clinical mastitis for vehicle control, CATH2 and its analogs.

FIG. 10 shows that treatment with CATH2 1-21L peptide demonstrated the greatest reduction in the bacterial load post challenge. Each analog tested showed a reduction in bacterial load as compared to the vehicle control.

FIG. 11 shows the least square mean somatic cell count.

FIG. 12 shows the analysis of fever for vehicle control, CATH2 and its analogs.

FIG. 13 shows the bovine serum albumin (BSA) levels in milk for vehicle control, CATH2 and its analogs.

FIG. 14 shows the milk haptoglobin levels for vehicle control, CATH2 and its analogs.

FIG. 15 shows the milk amyloid A levels for vehicle control, CATH2 and its analogs.

FIG. 16 shows the IL-1 beta levels for vehicle control, CATH2 and its analogs.

FIG. 17 shows the IL-6 levels for vehicle control, CATH2 and its analogs.

FIG. 18 shows the IL-8 levels for vehicle control, CATH2 and its analogs.

FIG. 19 shows the IL-10 levels for vehicle control, CATH2 and its analogs.

FIG. 20 shows the TNFα levels for vehicle control, CATH2 and its analogs.

FIG. 21 shows the results of Kaplan Meier analysis for vehicle control and CATH2 treatments.

FIG. 22 shows clinical mastitis incidence in a Streptococcus uberis challenge study.

### DETAILED DESCRIPTION OF THE INVENTION

The present subject matter may be understood more readily by reference to the following detailed description which forms a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

### Definitions

In the present disclosure the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment incudes from the one particular and/or to the other particular value. Similarly, when values are expressed as approximations, by use of the antecedent “about,” it is understood that the particular value forms another embodiment. All ranges are inclusive and combinable.

As used herein, the terms “component,” “composition,” “composition of compounds,” “compound,” “drug,” “pharmacologically active agent,” “active agent,” “therapeutic,” “therapy,” “treatment,” or “medicament” are used interchangeably herein to refer to a compound or compounds or composition of matter which, when administered to a subject (animal or human) induces a desired pharmacological and/or physiologic effect by local and/or systemic action.

As used herein, the terms “treatment” or “therapy” (as well as different forms thereof) include preventative (e.g., prophylactic), curative or palliative treatment. As used herein, the term “treating” includes alleviating or reducing at least one adverse or negative effect or symptom of a condition, disease or disorder. This condition, disease or disorder can be mastitis.

The terms “subject,” “individual,” and “patient” are used interchangeably herein, and refers to an animal to whom treatment, including prophylactic treatment, with the pharmaceutical composition according to the present invention, is provided. The term “subject” as used herein refers to human and non-human animals. The terms “non-human animals” and “non-human mammals” are used interchangeably herein and include all vertebrates, e.g., mammals, such as non-human primates, (particularly higher primates), bovine, sheep, goat, dog, cat, rodent, (e.g. mouse or rat), guinea pig, pig, rabbits, horses and non-mammals such as reptiles, amphibians, chickens, and turkeys.

### Cath2 Molecules

The invention provides CATH2 peptide and variants thereof. The inventors of the instant application have surprisingly and unexpectedly found that mastitis can be effectively treated by the administration of CATH2 peptide or its variants.

The term “peptide” as used herein, refers to a sequence of amino acids coupled by a peptide bond, wherein the amino acids are one of the twenty naturally peptide-building amino acids and wherein one or all of the amino acids can be in the L-configuration or in the D-configuration, or, for isoleucine and threonine in the D-allo configuration (only inversion at one of the chiral centers). A peptide according to the invention can be linear, i.e. wherein the first and last amino acids of the sequence have a free NH₂- or COOH-group respectively or are N-terminally (acetylation) and/or C-terminally (amidation) modified.

As used herein, the terms “CATH2” and “CMAP27” are used interchangeably. Like other members of the cathelicidin family CMAP27 is encoded as a prepropeptide (154 amino acids) and after proteolytic processing, a C-terminal peptide is released that has demonstrated potent broad-spectrum antimicrobial activity.

The amino acid sequence of this C-terminal peptide, called CMAP27 or CATH2, is RFGRFLRKIRRFRPKVTITIQGSARFG (SEQ ID NO.: 1) or its truncated functional sequence RFGRFLRKIRRFRPKVTITIQ (SEQ ID NO.: 35). The term, “CMAP27” or “CATH2,” as used herein, refers to either the 27 amino acid sequence set forth in SEQ ID NO.: 1 or the 21 amino acid sequence set forth in SEQ ID NO.: 35.

As used herein, a “CATH2 derivative” generally refers to a peptide that is a derivative of CATH2 in that it contains at least part of the sequence of CATH2 and that has maintained at least one antimicrobial properties of CATH2, although not necessarily to the same extent. In particular, antimicrobial activity against Gram (−) bacteria, Gram (+) bacteria, or a combination thereof is maintained.

As used herein, the term “variant” may refer to a structural or functional variant including, for example, analogs or derivatives of CATH2 peptide.

In one embodiment, the CATH2 derivative is selected from the group consisting of C-terminally and/or N-terminally truncated CATH2 derivatives, D-amino acid CATH2 derivatives, C-terminally or N-terminally truncated D-amino acid CATH2 derivatives, cyclic CATH2 derivatives and inverso and retroinverso CATH2-derivatives. The derivative or analog may contain one or more amino acid substitutions, preferably 1 to 5 amino acid substitutions, more preferably 1, 2, 3 or 4 amino acid substitutions. Preferably, the CATH2 derivative is selected from the group consisting of C-terminally and/or N-terminally truncated CATH2 derivatives, D-amino acid CATH2 derivatives and C-terminally or N-terminally truncated D-amino acid CATH2 derivatives, such as C-terminally or N-terminally truncated DCATH2. In one preferred embodiment, CATH2 or DCATH2 is used. DCATH2 may include the full length CATH2 peptide having D-amino acids.

“C-terminally truncated CATH2 derivatives” refers to truncated peptides lacking one or more amino acids at the C-terminus of CATH2, preferably lacking up to 17 amino acids, more preferably up to 12 amino acids, more preferably up to 6 amino acids. The examples are described in WO 2010/093245, which is incorporated herein by reference, and especially the peptides listed as CMAP26—NH₂, CMAP26, CMAP26 (P14→G), CMAP26 (P14→L), CMAP1-21, CMAP1-15, CMAP1-15 (F2→L), CMAP1-15 (F5→L), CMAP1-15 (F12→L), CMAP1-15 (3×F→L), CMAP1-15 (F2→W), CMAP1-15 (F5→W), CMAP1-15 (F12→W), CMAP1-15 (F2→W; F5→W; F12→W), CMAP1-13, CMAP1-12, CMAP1-11 and CMAP1-10 in Table 1 of said document and their acetylated and/or amidated derivatives. Herein, and in all amino acid sequence defined herein, the arrow notation indicates an amino acid substitution. For instance, F2→L indicates that the F at position 2 is replaced by L and F2, 5→W indicates that F at positions 2 and 5 is replaced by W. Further preferred are CMAP1-21 (F2→W), CMAP1-21 (F5→W), CMAP1-21 (F12→W), CMAP1-21 (F2, 5→W), CMAP1-21 (F5, 12→W), CMAP1-21 (F2, 12→W), CMAP1-21 (F2, 5, 12→W), CMAP1-21 (F2→Y), CMAP1-21 (F5→Y), CMAP1-21 (F12→Y), CMAP1-21 (F2,5→Y), CMAP1-21 (F5,12→Y), CMAP1-21 (F2, 12→Y), CMAP1-21 (F2, 5, 12→Y), CMAP1-21 (F2→W; F5→Y), CMAP1-21 (F2→Y; F5→W), CMAP1-21 (F5→W; F12→Y), CMAP1-21 (F5→Y; F12→W), CMAP1-21 (F2→W; F12→Y), CMAP1-21 (F2→Y; F12→W), CMAP1-21 (F2→W; F5→Y; F12→Y), CMAP1-21 (F2→Y; F5→W; F12→Y), and CMAP1-21 (F2→Y; F12→Y; F12→W). The examples of C-terminally truncated CATH2 derivatives are also described in WO2015/170984, which is incorporated herein by reference. The CMAP proteins identified above, may also be indicated as CATH2 peptides. CMAP1-21 then would be CATH2 (1-21).

“N-terminally truncated CATH2 derivatives” are CATH2 derivatives that are truncated at the N-terminal amino acid (arginine) of CATH2 thus lacking one or more amino acids at the N-terminus of CATH2, preferably lacking up to 10 amino acids, more preferably up to 7 amino acids, more preferably up to 6 amino acids. Examples of the N-terminally truncated CATH2 derivatives include, but not limited to, N-terminally truncated variants of CMAP1-21: CMAP4-21, CMAP5-21, CMAP6-21, CMAP7-21, CMAP8-21, CMAP9-21, CMAP10-21, CMAP11-21, CMAP4-21 (F5→W), CMAP4-21 (F5→Y), CMAP4-21 (F12→W), CMAP4-21 (F12→Y), CMAP4-21 (F5, F12→W), CMAP4-21 (F5, F12→Y), CMAP4-21 (F5→W, F12→Y), CMAP4-21 (F5→Y, F12→W), CMAP7-21 (F12→W), CMAP7-21 (F12→Y), CMAP10-21 (F12→W) and CMAP10-21 (F12→Y).

“D-amino acid CATH2 derivatives” are CATH2 derivatives as defined herein (including the above defined C- and N-terminally truncated CMAP27-derivatives) that contain at least one amino acid in the D configuration. A special category of these D-amino acid CATH2 derivatives are the peptides that are composed of only D amino acids (i.e. in which no L amino acid is present). This special category is herein defined as DCATH2. Also CATH2 itself, comprising one or more, or, alternatively, all D amino acids is comprised within this definition. In one embodiment, D-amino acid CATH2 derivatives are DCATH2. In some embodiments, the invention includes the following examples of D-amino acid CATH2 derivatives (indicated as D-C, and where all amino acids are in the D-form):

D-C(1-26)
(SEQ ID NO.: 17)
RFGRFLRKIRRFRPKVTITIQGSARF-NH₂
D-C(1-21)
(SEQ ID NO.: 18)
RFGRFLRKIRRFRPKVTITIQ-NH₂
D-C(4-21)
(SEQ ID NO.: 19)
RFLRKIRRFRPKVTITIQ-NH₂
D-C(7-21)
(SEQ ID NO.: 20)
RKIRRFRPKVTITIQ-NH₂
D-C(7-21)F/W
(SEQ ID NO.: 21)
RKIRRWRPKVTITIQ-NH₂
D-C(7-21)F/Y
(SEQ ID NO.: 22)
RKIRRYRPKVTITIQ-NH₂
D-C(10-21)F/W
(SEQ ID NO.: 23)
RRWRPKVTITIQ-NH₂
D-C(1-15)
(SEQ ID NO.: 24)
RFGRFLRKIRRFRPK-OH

In a particular embodiment, DCATH2 derivative is DCATH2 (1-21) (also called DC (1-21)) or DCATH2 (4-21) (also called DC (4-21)).

“Cyclic CATH2-derivatives” are CATH2 derivatives in which at least two non-adjacent amino acids are connected to form a ring structure. Although in principle any chemical binding construction may be used, such as replacing two non-adjacent amino acids in any of the above-mentioned CATH2 derivatives with a cysteine, where these cysteines then form an S-S bridge, a preferred binding system uses the binding between Bpg (Fmoc-L-bishomopropargylglycine) and an azido-resin, wherein the Bpg is attached to an internal arginine, leucine, phenylalanine or tryptophane residue and the azido-resin is attached to the C-terminal glutamic acid residue. Non-limiting examples such cyclic derivatives are below:

cycCMAP(1-21)[Lys8]
(SEQ ID NO.: 2)
RFGRFLR(Bpg)IRRFRPKVTITIQ(azido-resin)
cycCMAP(1-21)[Arg7]
(SEQ ID NO.: 3)
RFGRFL(Bpg)KIRRFRPKVTITIQ(azido-resin)
cycCMAP(1-21)[Leu6]
(SEQ ID NO.: 4)
RFGRF(Bpg)RKIRRFRPKVTITIQ(azido-resin)
cycCMAP(1-21)[Leu6],Phe2/Trp
(SEQ ID NO.: 5)
RWGRF(Bpg)RKIRRFRPKVTITIQ(azido-resin)
cycCMAP(1-21)[Leu6],Phe2,5/Trp
(SEQ ID NO.: 6)
RWGRW(Bpg)RKIRRFRPKVTITIQ(azido-resin)
cycCMAP(1-21)[Leu6],Phe2,5,12/Trp
(SEQ ID NO.: 7)
RWGRW(Bpg)RKIRRWRPKVTITIQ(azido-resin)
cycCMAP(1-21)[Leu6],Phe5,12/Trp
(SEQ ID NO.: 8)
RFGRW(Bpg)RKIRRWRPKVTITIQ(azido-resin)
cycCMAP(1-21)[Leu6],Phe12/Trp
(SEQ ID NO.: 9)
RFGRF(Bpg)RKIRRWRPKVTITIQ(azido-resin)

“Inverso” and “Retroinverso” CATH2 derivatives (“I”-CATH2 and “RI”-CATH2 derivatives) are peptides that have an inverted sequence with respect to the above-mentioned CATH2 derivatives, in the sense that the amino acids are connected to each other in a reverse order. When the inverted CATH2 derivatives contain one or more D amino acids they are termed “Retroinverso” or “RI”. If the inverted derivative only contains L-amino acids it is termed “Inverso” or “I”. The I and RI equivalent of CATH2 then may become GFRASGOITITVKPRFRRIKRLFRGFR (SEQ ID NO.: 10). Other non-limiting examples of such I or RI-CMAP27-derivatives are:

RI-C(1-21)
(SEQ ID NO.: 11)
QITITVKPRFRRIKRLFRGFR
RI-C(4-21)
(SEQ ID NO.: 12)
QITITVKPRFRRIKRLFR
RI-C(7-21)
(SEQ ID NO.: 13)
QITITVKPRFRRIKR
RI-C(7-21)F/W
(SEQ ID NO.: 14)
QITITVKPRWRRIKR
RI-C(7-21)F/Y
(SEQ ID NO.: 15)
QITITVKPRYRRIKR
RI-C(10-21)F/W
(SEQ ID NO.: 16)
QITITVKPRWRR

The I and RI-CMAP27 derivatives may be acetylated at their N-terminal and/or amidated at their C-terminal.

In a particular embodiment, the CATH2 or derivative thereof used in any method or use of the invention is CATH2, DCATH2, DCATH2 (1-21), DCATH2 (4-21), CMAP4-21, CMAP5-21, CMAP6-21, CMAP7-21, CMAP8-21, CMAP9-21, CMAP10-21, CMAP11-21, CMAP4-21 (F5→W), CMAP4-21 (F5→Y), CMAP4-21 (F12→W), CMAP4-21 (F12→Y), CMAP4-21 (F5, F12→W), CMAP4-21 (F5, F12→Y), CMAP4-21 (F5→W, F12→Y), CMAP4-21 (F5→Y, F12→W), CMAP7-21 (F12→W), CMAP7-21 (F12→Y), CMAP10-21 (F12→W) or CMAP10-21 (F12→Y). In some embodiments, the CATH2 or derivative thereof used in any method or use of the invention is CATH2, DCATH2, DCATH2 (1-21) or DCATH2 (4-21). In one embodiment, the CATH2 or derivative thereof used in any method or use of the invention is DCATH2, DCATH2 (1-21) or DCATH2 (4-21).

In some embodiments, the CATH2 or derivative thereof used in any method or use of the invention is one or more the peptides below.

(SEQ ID NO.: 25)
RCGRFLRKIRPFRRKVTITRQ
(SEQ ID NO.: 26)
RCGRFLRKIRPFRGKVTITRQ
(SEQ ID NO.: 27)
RFGRFLRKIRRFRGKVTITRQ
(SEQ ID NO.: 28)
RWGRWLRKIRRWRPKVTITRQ
(SEQ ID NO.: 29)
RWGRWLRKIRRWRPKVTITIQ
(SEQ ID NO.: 30)
RFLRKIRRFRPKVTITRQ
(SEQ ID NO.: 31)
RFLRKIRRFRGKVTITRQ
(SEQ ID NO.: 32)
RWLRKIRRWRPKVTITIQ
(SEQ ID NO.: 33)
RWLRKIRRWRPKVTITRQ
(SEQ ID NO.: 34)
RWLRKIRRWRGKVTITRQ
(SEQ ID NO.: 35)
RFGRFLRKIRRFRPKVTITIQ
(SEQ ID NO.: 36)
RCGRFLRKIRPFRRKVTITCQ
(SEQ ID NO.: 37)
RFGRWLRKIRRYRGKVTITIQ

In one exemplary embodiment, the peptide of the invention is an immune modulatory peptide having no antibiotic activity because CATH2's intrinsic antimicrobial activity is abrogated with milk or milk proteins. In another exemplary embodiment, the peptide of the invention is an immune modulatory peptide having no direct killing effect on bacteria because CATH2's intrinsic antimicrobial activity is abrogated with milk or milk proteins.

### Methods for Producing Peptides

Methods for producing peptides are well known in the art and fully described in U.S. Patent Application Publication 20170145065, which is incorporated by reference herein in its entirety. Any suitable method can be used for making the peptides of the invention. In one embodiment, the peptides of the invention are produced synthetically. Peptide chemical synthesis techniques are well known in the art and fully described in, for example, U.S. Patent Application Publication 20170145065 and Merrifield, 1963, J. Am. Chem. Soc., vol. 85, pages 2149-2154, which are incorporated by reference herein. Peptides may be isolated from the reaction mixture by chromatographic methods, such as reverse-phase HPLC.

In another embodiment, the peptides of the invention the peptides of the invention are produced recombinantly by methods well known in the art. For example, peptides may be produced by recombinant DNA techniques by cloning and expressing within a host micro-organism or cell a DNA fragment carrying a nucleic acid sequence encoding one of the above-described peptides. Nucleic acid coding sequences can be prepared synthetically, or may be derived from existing nucleic acid sequences (e.g. the sequence coding for wild-type CATH2) by site-directed mutagenesis. These nucleic acid sequences may then be cloned in a suitable expression vector and transformed or transfected into a suitable host cell, such as Escherichia coli, Bacillus spp, Lactobacillus spp, Streptomyces spp, mammalian cells (such as CHO, HEK or COS-1 cells), yeasts (e.g. Saccharomyces, Schizophyllum), insect cells or viral expression systems, such as baculovirus systems, or plant cells. Techniques of constructing and expressing the nucleic acids are well known to a person skilled in the art.

Peptides can be isolated from the culture of the host cells. This can be achieved by common protein purification and isolation techniques, which are available in the art. Such techniques may e.g. involve immunoadsorption or chromatography. Peptides can also be provided with a tag (such as a histidine tag) during synthesis, which allows for a rapid binding and purification, after which the tag is enzymatically removed to obtain the active peptide.

Alternatively, the peptides can be produced in cell-free systems, such as the Expressway cell-free system of Invitrogen.

### Pharmaceutical Compositions

In another embodiment, provided herein is a pharmaceutical composition to treat a mastitis in a subject, the composition comprising: a therapeutically effective amount of CATH2 peptide or a variant thereof, wherein said CATH2 peptide or said variant thereof is present in an amount effective to treat mastitis.

The invention also provides a pharmaceutical composition comprising the peptide of the invention and one or more pharmaceutically acceptable carriers. “Pharmaceutically acceptable carriers” include any excipient which is nontoxic to the cell or mammal being exposed thereto at the dosages and concentrations employed. The pharmaceutical composition may include one or additional therapeutic agents.

Pharmaceutically acceptable carriers include solvents, dispersion media, buffers, coatings, antibacterial and antifungal agents, wetting agents, preservatives, buggers, chelating agents, antioxidants, isotonic agents and absorption delaying agents.

Pharmaceutically acceptable carriers include water; saline; phosphate buffered saline; dextrose; glycerol; alcohols such as ethanol and isopropanol; phosphate, citrate and other organic acids; ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; EDTA; salt forming counterions such as sodium; and/or nonionic surfactants such as TWEEN, polyethylene glycol (PEG), and PLURONICS; isotonic agents such as sugars, polyalcohols such as mannitol and sorbitol, and sodium chloride; as well as combinations thereof. Antibacterial and antifungal agents include parabens, chlorobutanol, phenol, ascorbic acid, and thimerosal.

The pharmaceutical compositions of the invention may be formulated in a variety of ways, including for example, liquid, semi-solid and solid dosage forms, such as liquid solutions (e.g., injectable and infusible solutions), dispersions or suspensions, tablets, pills, powders, liposomes and suppositories. In some embodiments, the compositions are in the form of injectable or infusible solutions. The composition is in a form suitable for oral, intravenous, intraarterial, intramuscular, subcutaneous, parenteral, transmucosal, transdermal, or topical administration. The composition may be formulated as an immediate, controlled, extended or delayed release composition.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the subject invention, pharmaceutically acceptable carriers include, but are not limited to, 0.01-0.1M and preferably 0.05M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases and the like.

More particularly, pharmaceutical compositions suitable for injectable or infusible use, for example, intra-mammary injectable or infusible use, include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Suitable formulations for use in the therapeutic methods disclosed herein are described in Remington's Pharmaceutical Sciences, Mack Publishing Co., 16th ed. (1980).

In some embodiments, the composition includes isotonic agents, for example, sugars, polyalcohols, such as mannitol, sorbitol, or sodium chloride. Prolonged absorption of the injectable compositions can be brought about by including in the composition an agent which delays absorption, for example, aluminum monostearate and gelatin.

Sterile injectable solutions can be prepared by incorporating the molecule, by itself or in combination with other active agents, in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, one method of preparation is vacuum drying and freeze-drying, which yields a powder of an active ingredient plus any additional desired ingredient from a previously sterile-filtered solution thereof. The preparations for injections or infusions are processed, filled into containers such as ampoules, bags, bottles, syringes or vials, and sealed under aseptic conditions according to methods known in the art. Further, the preparations may be packaged and sold in the form of a kit such as those described in US Appl. Publ. No. 2002/0102208 A1, which is incorporated herein by reference in its entirety. Such articles of manufacture will preferably have labels or package inserts indicating that the associated compositions are useful for treating a subject suffering from, or predisposed to mastitis associated diseases or disorders.

Effective doses of the compositions of the present invention, for treatment of conditions or diseases as described herein vary depending upon many different factors, including means of administration, target site, physiological state of the patient, whether the patient is human or an animal, other medications administered, and whether treatment is prophylactic or therapeutic. Usually, the patient is a non-human mammal (e.g., a cow), but humans can also be treated. Treatment dosages may be titrated using routine methods known to those of skill in the art to optimize safety and efficacy.

The pharmaceutical compositions of the invention may include a “therapeutically effective amount.” A “therapeutically effective amount” refers to an amount effective, at dosages and for periods of time necessary, to achieve the desired therapeutic result. A therapeutically effective amount of a molecule may vary according to factors such as the disease state, age, sex, and weight of the individual (e.g., animal), and the ability of the molecule to elicit a desired response in the individual. A therapeutically effective amount is also one in which any toxic or detrimental effects of the molecule are outweighed by the therapeutically beneficial effects.

The invention further provides a kit comprising a therapeutically effective amount of a CATH2 peptide, or a derivative thereof.

The invention further provides methods of treating a disease or condition, comprising administering to a mammal in need thereof a therapeutically effective amount of a CATH2 peptide, or a derivative thereof.

As used herein, the terms “treat” and “treatment” refer to therapeutic treatment, including prophylactic or preventative measures, wherein the object is to prevent or slow down (lessen) an undesired physiological change associated with a disease or condition. Beneficial or desired clinical results include, but are not limited to, alleviation of symptoms, diminishment of the extent of a disease or condition, stabilization of a disease or condition (i.e., where the disease or condition does not worsen), delay or slowing of the progression of a disease or condition, amelioration or palliation of the disease or condition, and remission (whether partial or total) of the disease or condition, whether detectable or undetectable. “Treatment” can also mean prolonging survival as compared to expected survival if not receiving treatment. Those in need of treatment include those already with the disease or condition as well as those prone to having the disease or condition or those in which the disease or condition is to be prevented.

Mastitis which can be treated by the invention include any clinical or pre-clinical mastitis. Mastitis occurs when the udder or breast tissue becomes inflamed. Inflammation may be caused by many types of injury including infectious agents and their toxins, physical trauma or chemical irritants. Many microorganisms or bacteria have been identified as causing mastitis. In one embodiment, mastitis is caused by one or more of pathogens, including, for example, but not limited to, Staphylococcus aureus, Streptococcus agalactiae, Streptococcus dysgalactiae, Streptococcus uberis and E. coli.

In some embodiment, mastitis is associated with one or more pathogens, including, for example, but not limited to, E. coli, Klebsiella spp., Enterobacter spp., Salmonella spp., Citrobacter spp., Serratia spp., Shigella spp., Edwardsiella spp., Hafnia spp., Morganella spp., Providencia spp., Yersinia spp., Staphylococcus aureus, Staphylococcus spp., Pseudomonas Streptococcus agalactiae, Streptococcus dysgalactiae, Streptococcus uberis, spp., Streptococcus spp., Enterococci, Corynebacterium spp., Arcanobacterium spp., Actinomyces spp., Mycobacterium spp., Prototheca spp., Mycoplasma spp., and Erwinia spp.

Mastitis may cause compositional changes in milk, including an increase in somatic cell count (SCC). In bovine, milk from normal (uninfected) cows generally contain below 200,000 somatic cells/ml. An elevation in SCC, above 300,000 somatic cells/ml is abnormal and is an indication of inflammation of the udder. In one embodiment, the clinical mastitis includes SCC above approximately 300,000 somatic cells/ml in milk. In another embodiment, the sub-clinical mastitis includes SCC in the range from about 200,000 to about 300,000 somatic cells/ml in milk.

Protein breakdown in the milk can occur in milk from cows with clinical or subclinical mastitis due to the presence of proteolytic enzymes.

More than one agent may be administered, either incorporated into the same composition or administered as separate compositions.

The peptide of the invention may be administered alone, or in combination with one or more therapeutically effective agents (e.g., an antibiotic, another immunomodulator, another cathelicidin, or a combination thereof) or treatments. The other therapeutically effective agent may be conjugated to the peptide of the invention, incorporated into the same composition as the peptide of the invention, or may be administered as a separate composition. The other therapeutically agent or treatment may be administered prior to, during and/or after the administration of the peptide of the invention.

In one embodiment, the peptide of the invention is co-administered with another therapeutic agent. In another embodiment, the peptide of the invention is administered independently from the administration of another therapeutic agent. In one embodiment, the peptide of the invention is administered first, followed by the administration of another therapeutic agent. In another embodiment, another therapeutic agent is administered first, followed by the administration of the peptide of the invention.

The administration of the peptide of the invention with other agents and/or treatments may occur simultaneously, or separately, via the same or different route, at the same or different times. Dosage regimens may be adjusted to provide the optimum desired response (e.g., a therapeutic or prophylactic response).

In one example, a single bolus may be administered. In another example, several divided doses may be administered over time. In yet another example, a dose may be proportionally reduced or increased as indicated by the exigencies of the therapeutic situation. Dosage unit form, as used herein, refers to physically discrete units suited as unitary dosages for treating mammalian subjects. Each unit may contain a predetermined quantity of active compound calculated to produce a desired therapeutic effect. In some embodiments, the dosage unit forms of the invention are dictated by and directly dependent on the unique characteristics of the active compound and the particular therapeutic or prophylactic effect to be achieved.

The composition of the invention may be administered only once, or it may be administered multiple times. For multiple dosages, the composition may be, for example, administered three times a day, twice a day, once a day, once every two days, twice a week, weekly, once every two weeks, or monthly.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

“Administration” to a subject is not limited to any particular delivery system and may include, without limitation, parenteral (including intramammary, subcutaneous, intravenous, intramedullary, intraarticular, intramuscular, or intraperitoneal injection) rectal, topical, transdermal or oral (for example, in capsules, suspensions or tablets). Administration to a host may occur in a single dose or in repeat administrations, and in any of a variety of physiologically acceptable salt forms, and/or with an acceptable pharmaceutical carrier and/or additive as part of a pharmaceutical composition (described earlier). Once again, physiologically acceptable salt forms and standard pharmaceutical formulation techniques are well known to persons skilled in the art (see, for example, Remington's Pharmaceutical Sciences, Mack Publishing Co.).

The composition of the invention (e.g., CATH2 peptide) may be administered parenterally (e.g., intramammary, intravenous, subcutaneous, intraperitoneal, intramuscular). In a particular embodiment, the composition of the invention is administered by intramammary infusion or injection.

In another aspect, the invention provides an intra-mammary delivery composition comprising: CATH2 peptide or a variant thereof. In one example, the peptide or its variant is present in the composition in an amount effective to treat mastitis in a subject.

The composition of the invention may also be administered by intramuscular or subcutaneous injection. In some embodiments, the composition of the invention may be administered orally. As used herein, a “composition” refers to any composition that contains a pharmaceutically effective amount of one or more active ingredients (e.g., a CATH2 peptide or a derivative thereof).

In yet another aspect, the invention provides a kit or a mammary delivery device comprising: a chamber for storing a composition, wherein said composition comprises CATH2 peptide or a variant thereof.

Mammary delivery devices, including intra-mammary delivery devices, are well known in the art. In one embodiment, the device of the invention is an intra-mammary infusion device. In another embodiment, the device of the invention is a syringe. In yet another embodiment, the device of the invention is a teat-sealant device.

The inventions described herein can be used to treat any suitable mammal, including primates, such as bovine (e.g., cow, buffalo, bison, yak), goat, sheep, horses, cats, dogs, monkeys, humans, rabbits, and rodents such as rats and mice. In one embodiment, the mammal to be treated is bovine.

All patents and literature references cited in the present specification are hereby incorporated by reference in their entirety.

The following examples are provided to supplement the prior disclosure and to provide a better understanding of the subject matter described herein. These examples should not be considered to limit the described subject matter. It is understood that the examples and embodiments described herein are for illustrative purposes only and that various modifications or changes in light thereof will be apparent to persons skilled in the art and are to be included within, and can be made without departing from, the true scope of the invention.

### EXAMPLES

### Example 1

### Treating Mastitis by CATH2

Our results demonstrate that the Chicken Cathelicidin 2 (CATH2) peptide drives immune response towards resolution of infection and inflammation. Thus, it can effectively treat early infections and reduce the severity of existing infections and inflammation. The impact of driving immune-mediated resolution ultimately complements the existing antibiotic therapeutics in severe cases, where the traditional antibiotic treatment will target clearance of the invading bacteria and CATH2 will aid in the resolution of the associated immunopathology.

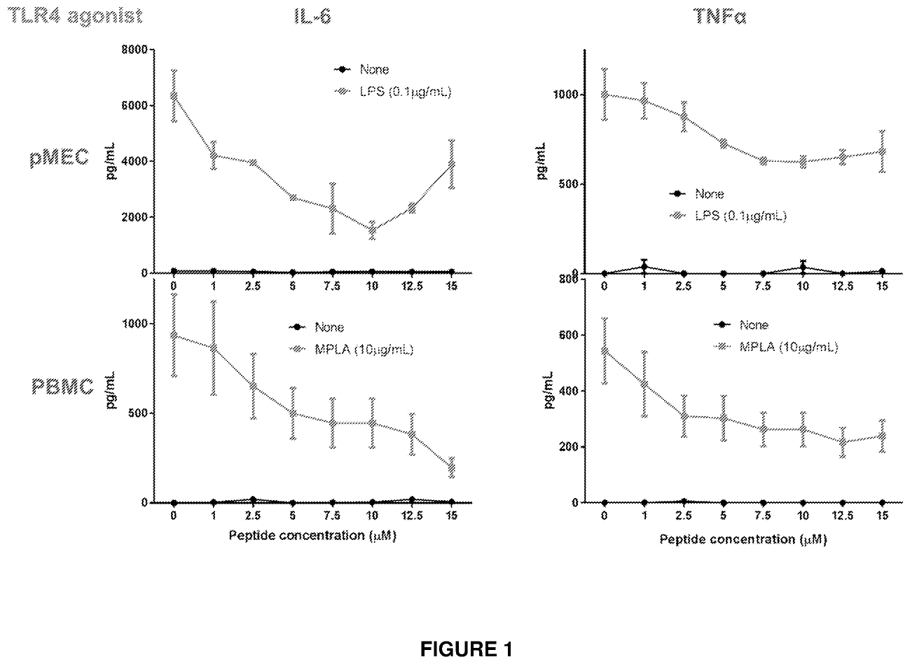

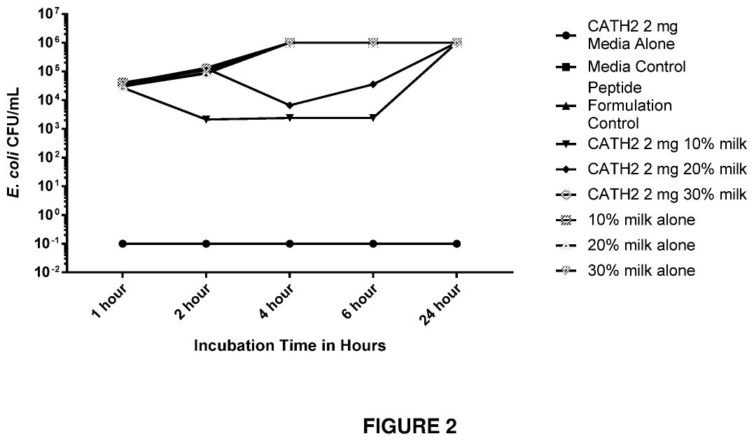

Chicken CATH2 is a host defense peptide that is naturally expressed in chicken heterophils and several tissues. The inventors of the instant application have demonstrated that a truncation of this peptide, specifically a 1-21 amino acid sequence of the C-terminal active region of the full-length pro-peptide, administered by the intramammary route in lactating cattle, effectively inhibits Escherichia coli mastitis. In vitro studies demonstrated a phenotypic response with exposure to varying concentrations of the peptide resulting in minor increase in proinflammatory cytokines in healthy unstimulated bovine mammary epithelial cells. Interestingly, upon lipopolysaccharide (LPS) or monophosphoryl lipid A (MPLA) stimulation, exposure to the peptide results in a dose-dependent inhibition of proinflammatory cytokines (FIG. 1). LPS and MPLA serve as potent Toll-like Receptor 4 (TLR 4) agonists in this assay and induce high levels of proinflammatory cytokines such as interleukin 6 (IL-6) and tumor necrosis factor alpha (TNFα). CATH2 effectively inhibits these proinflammatory cytokines in vitro in primary bovine mammary epithelial cells (pMEC) and bovine peripheral blood mononuclear cells (PBMCs), demonstrating a clinical phenotype that is highly consistent with the desirable therapeutic activity towards inflammatory mechanisms in mastitis.

E. coli Challenge Study

### In Vivo Efficacy

A frozen stock of E. coli was used to prepare a suspension of approximately 500 colony forming units/10 mL using standard site procedures. Standard plate counts were conducted on the challenge suspension immediately after preparation and after all animals were challenged. Details of the challenge inoculum preparation including concentration (CFU/10 mL) for the pre- and post-challenge preparation were documented and confirmed to be in range. Locally sourced healthy adult female Holstein Crossbred Cattle in the first, second, or third lactation were enrolled into the studies. Animals challenged with E. coli were susceptible to E. coli as determined by vaccination records and low serum titer to E. coli. Animals enrolled were confirmed to be between 14 and 35 days post-calving at enrollment. Animals had a somatic cell count (SCC)≤200,000 per mL at time of enrollment. Following challenge quarters bacteriologically positive for E. coli were regarded as infected. Clinical mastitis was defined as a challenged quarter having a clinical score of at least 1 for either milk appearance or udder evaluation and an isolation of E. coli from the challenged quarter. Subclinical mastitis was defined as isolation of E. coli from the challenged quarter and elevated SSC in the absence of abnormal milk or udder score. This isolate elicits a moderate case of E. coli mastitis with body temperature increase in the first 24 hours post challenge and consistent local signs on udder appearance and milk quality.

In this study, the primary objective was to evaluate efficacy of CATH2 in preventing or reducing clinical severity in a lactating cow E. coli mastitis challenge model. The CATH2 peptide was administered by intramammary infusion to cows less than 35 days in lactation. Post-challenge, the CATH2 induced a significant reduction in intramammary infection and clinical mastitis. The investigators evaluated multiple endpoints to understand the impact on infection including bacterial load, clinical observations, differential somatic cell counts, and cytokine analysis. The results on all endpoints show that CATH2 had an impact on the resolution of inflammation and infection. This study demonstrates the proof of concept for efficacy in the target animal with a relevant mastitis pathogen. The data presented below provide confidence in the capacity of the peptide to modulate the host immune response in prevention of clinical mastitis.

### Efficacy Study

In this study, animals were treated with 50 mg/10 mL at 12-hour intervals starting 12 hours prior to E. coli challenge. A total of 4 doses were administered by intramammary infusion. Dose administration concluded 24 hours after challenge. The objective of the study was to evaluate the potential for CATH2 to reduce the severity of clinical mastitis by modulation of the host immune response. The dose regimen was designed to ensure sufficient exposure in the absence of understanding the duration of the immunological effect driven by the peptide in cattle.

The experimental design was a randomized design with a one-way treatment structure replicated in 3 batches. The success criteria for efficacy for immunomodulation was ≥40% of the treatment group to demonstrate a reduction in somatic cell count (SCC) and fever, improved udder and attitude scores and reduction of bacterial counts. Milk samples were collected for transcriptomic analysis and cytokine profiles in the whey at 12 hours post-challenge. Differential quantitative milk leukocyte counts were collected for 7 days post challenge.

TABLE 1
Study Design for Evaluation of 1-21 L Chicken Cathelicidin 2

| | Number of | Challenge | Treatment | | Route of |
|---|---|---|---|---|---|
| Treatment | Animals | Administration | Description/Dose | Dosage | Administration |
| T01 | 23 | Escherichia coli, | Saline | 4 doses, at −12, 0, | Intramammary |
| | | Day 0 | | 12, and 24 hours | Infusion |
| | | | | respective of | |
| | | | | challenge | |
| T03 | 23 | | CATH2 in aqueous | 4 doses, at −12, 0, | |
| | | | formulation; | 12, and 24 hours | |
| | | | 50 mg/10 mL | respective of | |
| | | | | challenge | |

This E. coli challenge model is highly acute, presenting with predictable increases in somatic cell counts and body temperature, poor milk quality, and clinical udder scores. Corresponding with the clinical signs of disease, the bacterial load increases during the first 24 hours post challenge. The animals are able to clear the bacterial challenge within approximately 7 days. Clinical signs of inflammation can persist beyond the Day 7 time point. At the time of the study the duration of effect needed to achieve proof of concept efficacy in this model system was not well understood. Therefore, (CATH2) was dosed 4 times to maintain exposure of the peptide during the onset of the infection. The dose concentration was selected based transcriptomics analysis in a small biomarker study, that provided an indication of a response consistent with resolution of inflammation as a result of an E. coli infection.

FIG. 3 demonstrates animals treated with the saline control show high susceptibility to the challenge isolate with 95.65% animals showing signs consistent with clinical mastitis. In contrast, CATH2 treatment resulted in only 8.70% of the animals showing signs of clinical mastitis.

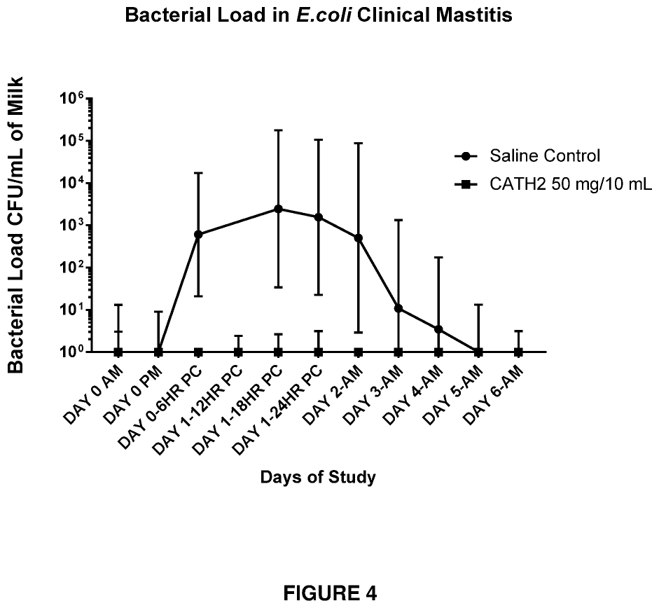

FIG. 4 shows the bacterial load in the milk measured at different time points over the first 6 days of the study. The bacterial challenge inoculum was administered Day 0. The results demonstrated a significant inhibition of E. coli ability to establish infection in the CATH2-treated animals. Clinical mastitis was defined as a challenged quarter having a clinical score of at least 1 for either milk appearance or udder evaluation, with isolation of E. coli from the challenged quarter. A total of 21 out of 23 (91.3%) CATH2-treated animals never met criteria for clinical mastitis, as compared to the Saline control, where 22 of 23 (95.65%) met criteria for clinical mastitis. Intramammary infection was defined as isolation of E. coli from the challenged quarter. A total of 20 of the 23 (86.96%) animals were never positive for intramammary infection in the CATH2 group as compared to the Saline control, with 22 of 23 (95.65%) positive for intramammary infection. Subclinical mastitis was defined as isolation of E. coli from the challenged quarter and elevated SCC (>200,000 cells/mL) in the absence of abnormal milk or udder score. None of the CATH2-treated animals qualified for subclinical mastitis, whereas 20 of 23 animals in the Saline control group met criteria for subclinical mastitis at some point in the 14-day study. These data demonstrate the ability for CATH2 to inhibit establishment of E. coli infection and prevent clinical and subclinical mastitis.

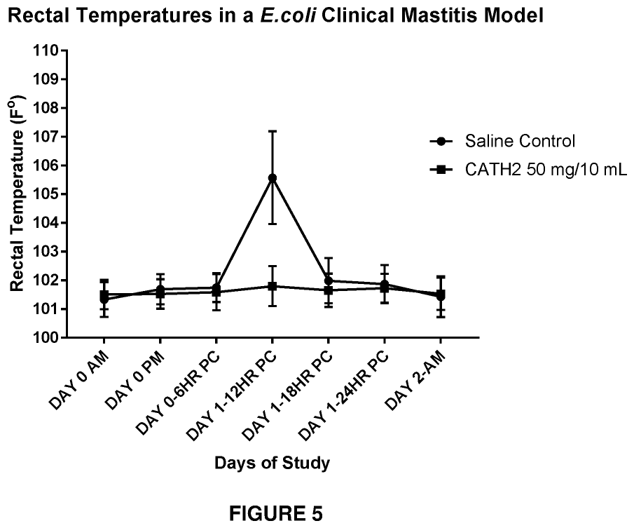

A consistent characteristic of this E. coli challenge model in lactating cows is a significant rise in body temperature 12-18 hours post-challenge administration. In FIG. 5, the rectal temperatures clearly showed this characteristic temperature spike at 12 hours post-challenge for treatment group Saline. However, CATH2-treated animals maintained normal body temperatures throughout the study, with 22 of 23 (95.7%) animals not showing an increase in body temperature as compared to the Saline-treated animals, in which 20 of 23 (87%) animals showed an elevated body temperature.

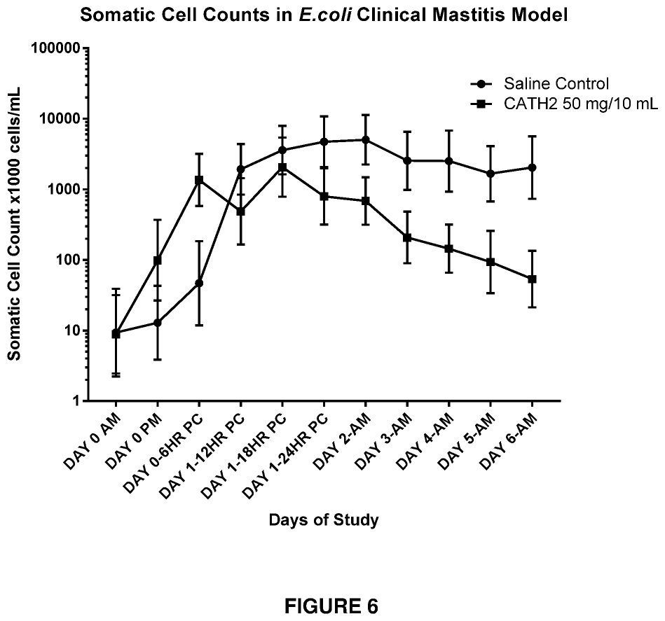

FIG. 6 shows the somatic cell count (SCC) for the saline control and CATH2 treated animals. There is a notable increase in the SCC at 6 hours post challenge in the CATH2 treated animals. With the rapid increase in SCC observed in CATH2 treated animals, the bacterial load is managed in these animals by enhanced bacterial clearance responses, which is supported by transcriptomics analysis.

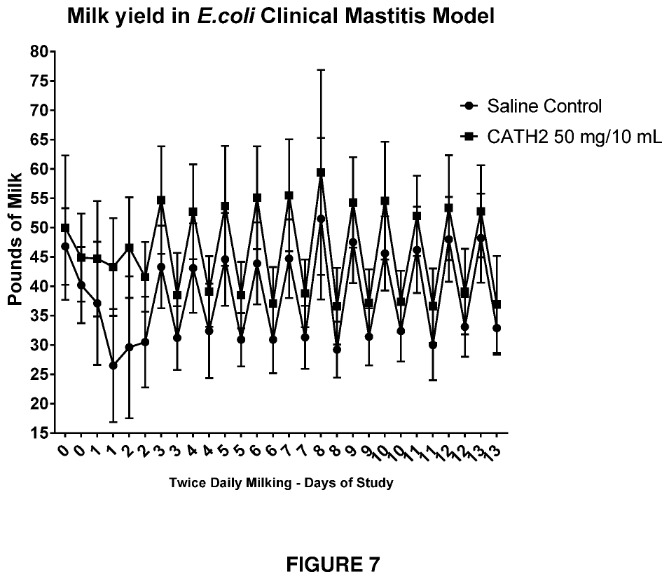

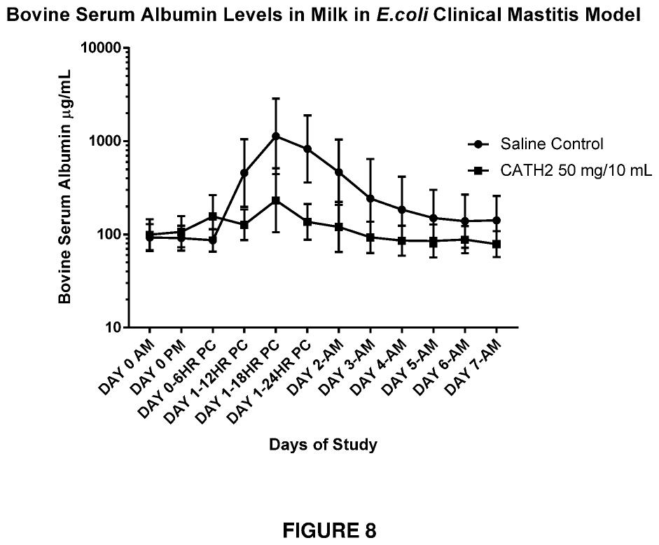

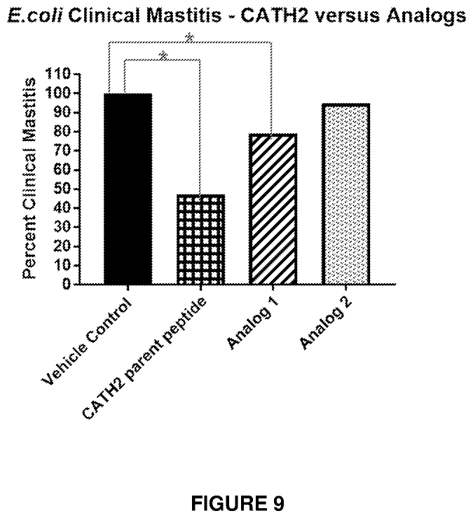

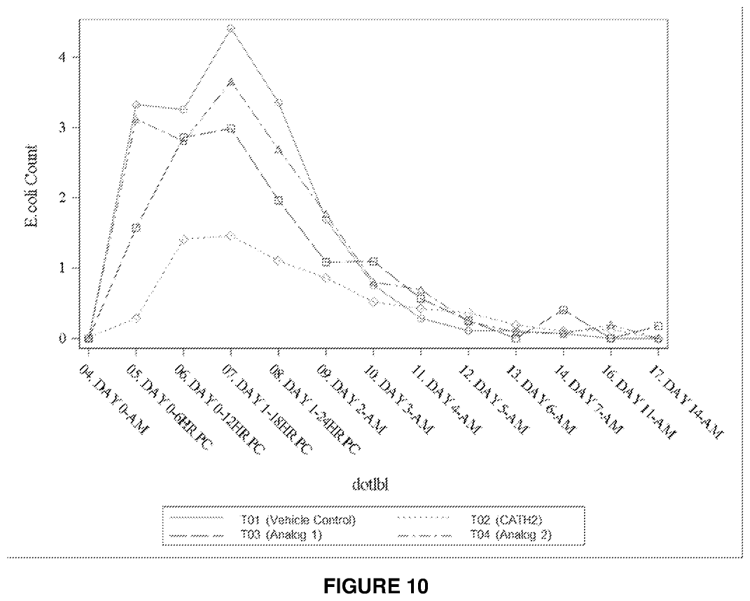

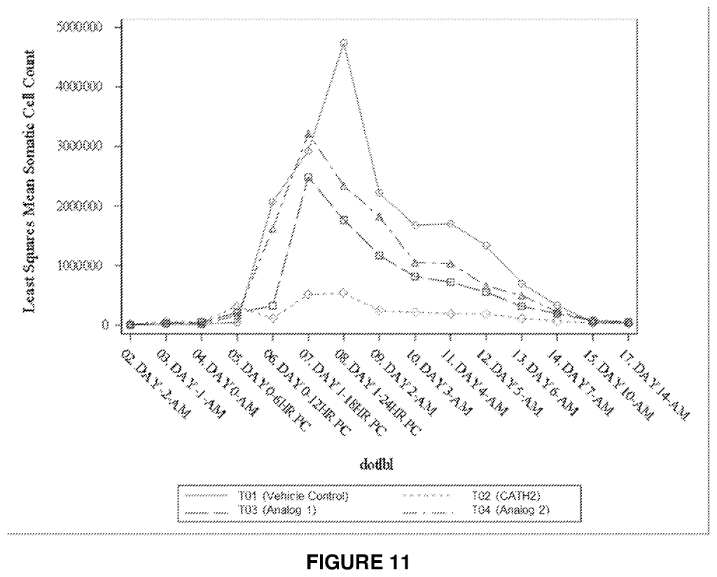

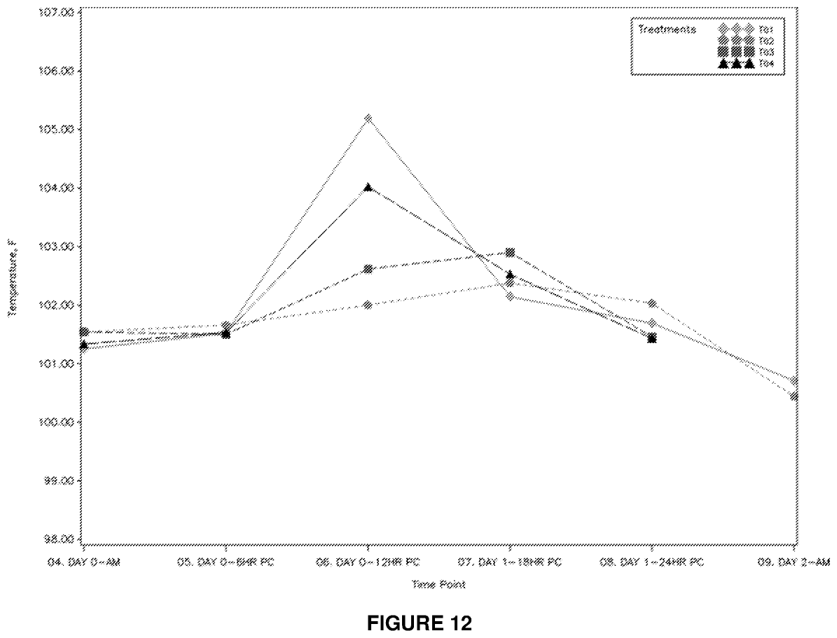

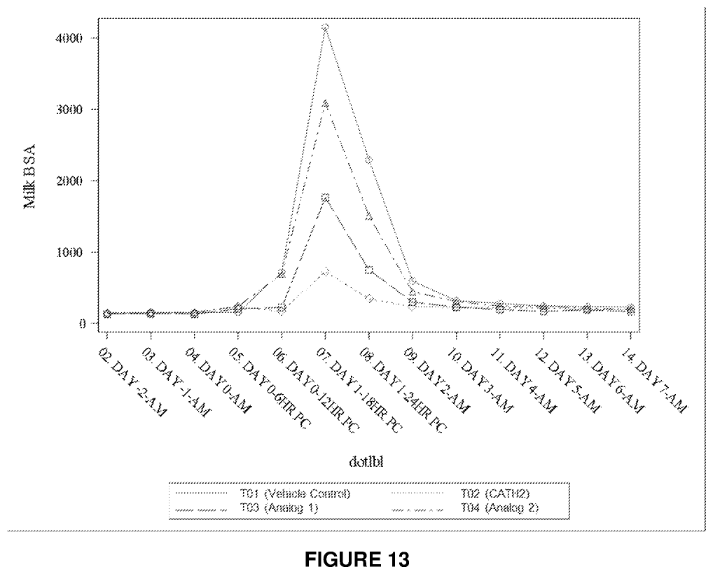

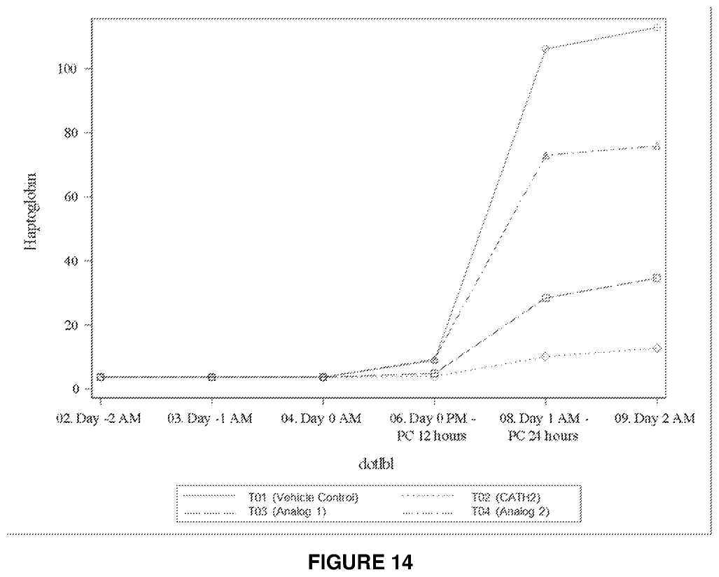

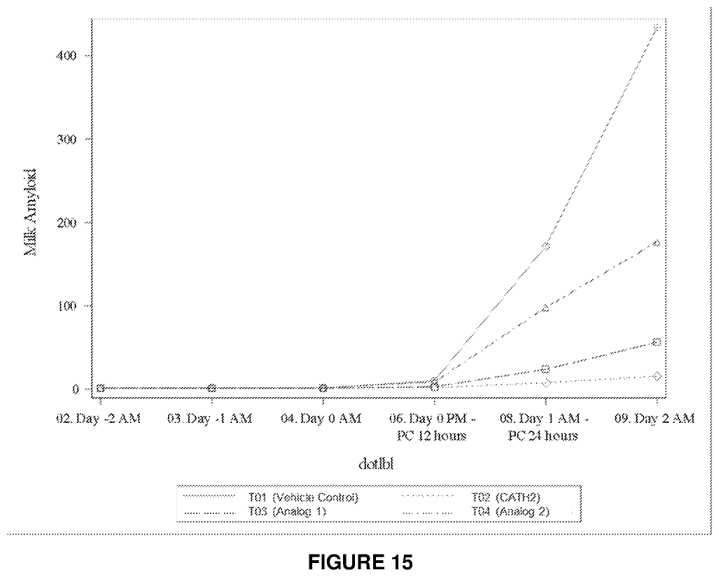

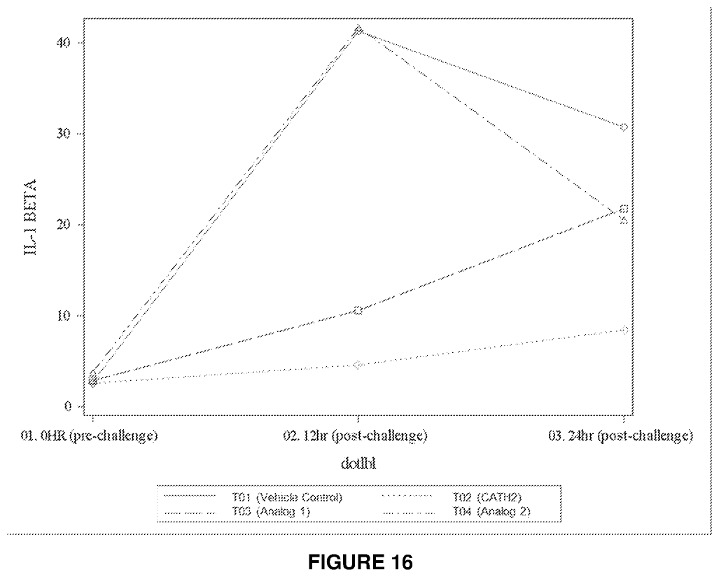

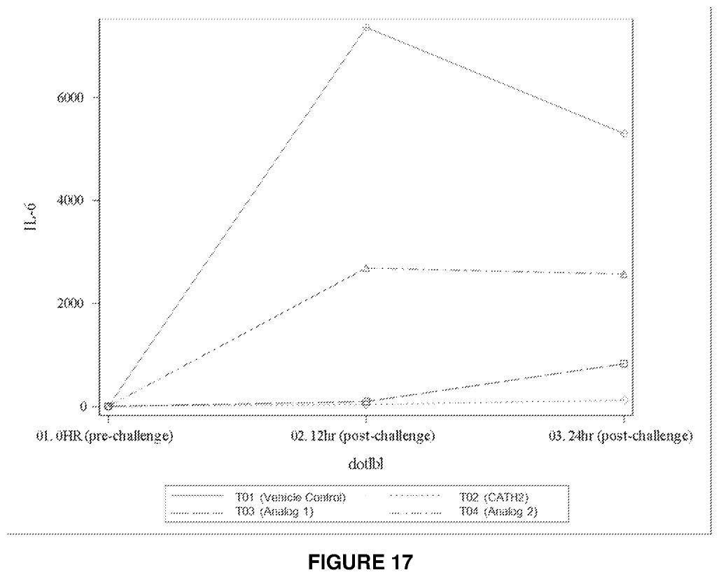

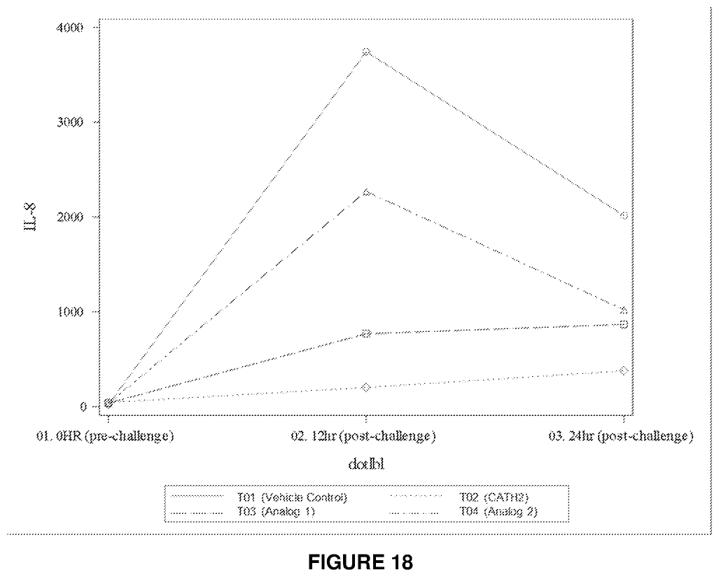

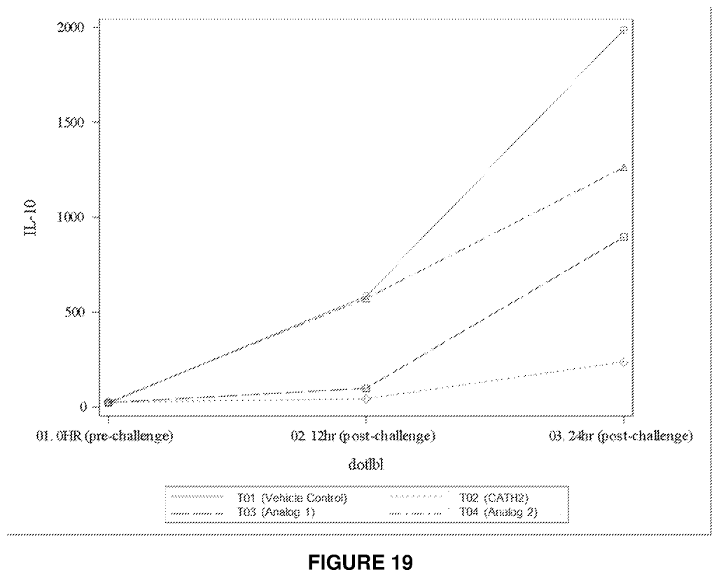

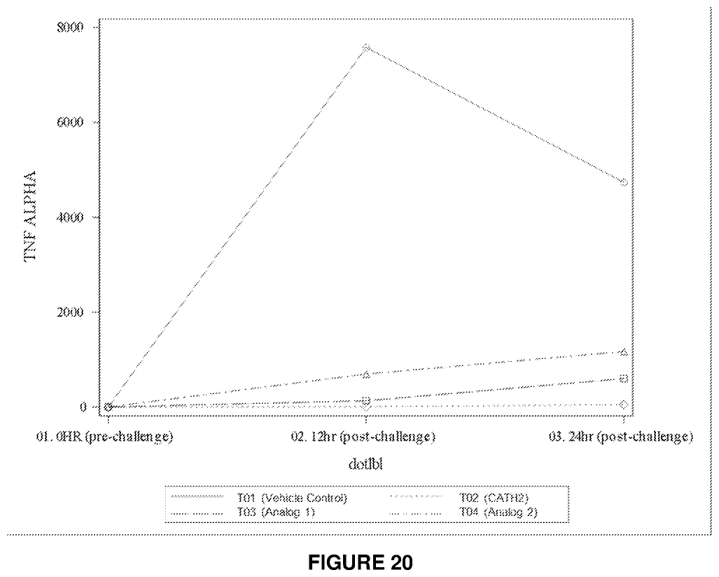

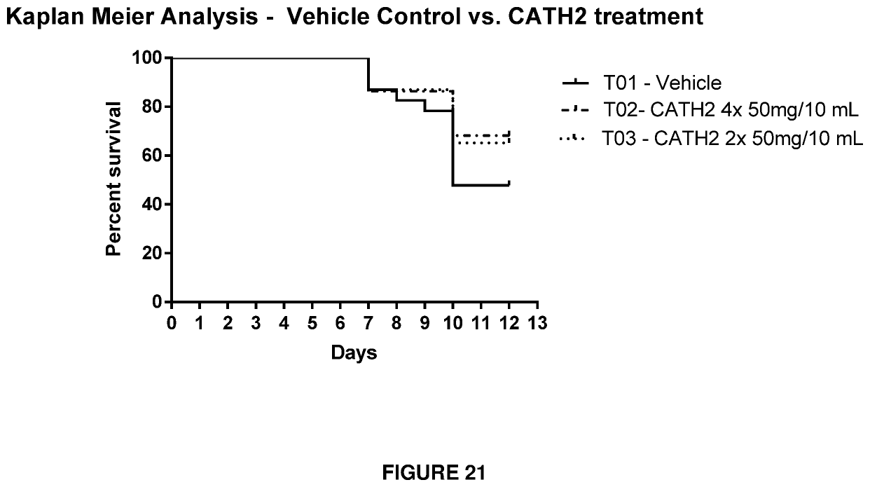

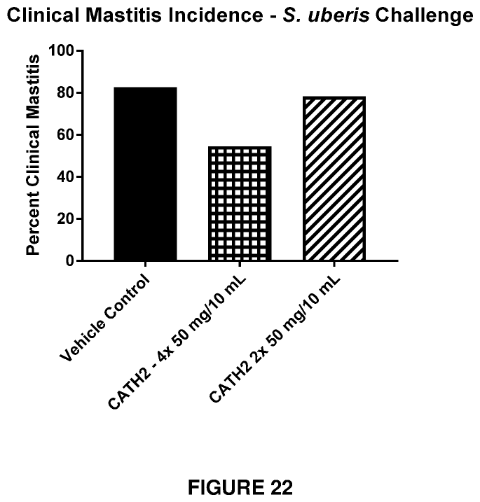

FIG. 7 shows the milk yields at each milking for each study day. The AfiLab® system is an in-line, real time, milk analysis of yield and milk components. Following challenge administration at Study Day 0 following milking, the milk yield for all treatment groups is reduced as compared to CATH2-treated animals, where the mean milk yield at each milking remains high. On Study Day 0 and 1, to accommodate drug dosing, monitoring of the infection, and sample collection, the milking schedule was set to exactly 12 hours apart from the start of the study. Beginning on Study Day 2, the milking cycle procedure returned to normal where milkings occurred 10 hours to 14 hours apart. The timing of milking is reflected in the graph with the higher peaks of milk yield occurring at the morning milking, which would have been approximately 14 hours post the previous afternoon milking. Throughout the study the CATH2 treated animals maintained high milk yields demonstrating overall good health. Milk yield is a highly significant parameter for return on investment for the dairy producer.

Bovine Serum Albumin (BSA) levels in milk are a surrogate indicator of mammary inflammation and damage to mammary epithelium. Historically, BSA levels in milk have been measured in this model for this purpose. FIG. 8 shows analysis of milk BSA from the challenged quarter; plot of treatment least square means. CATH2-treated animals had only modest increases in BSA levels over baseline as compared to all other treatment groups. CATH2-treated animals had statistically significantly reduced BSA levels in the milk when compared with Saline control at all time points after Day 0 at 6 hours post-challenge, with a return to baseline levels by Study Day 3. The saline control group does not return to baseline levels by Study Day 7. BSA measurement was not carried out past Study Day 7.

At 12 hours post-challenge, milk samples were collected for the transcriptomics analysis and cytokine profiling in the whey. At this time point, Saline-treated animals were at the peak of the temperature spike. Based on previous studies to characterize the response to E. coli in the model, the 12-hour post challenge timepoint also corresponded with peak cytokine production. The CATH2-treated animals showed significantly lower levels of key inflammatory cytokines as compared to Saline. These data correlate with the in vitro phenotypic analysis with primary bovine mammary epithelial cells where a CATH2 mediated inhibition of IL-6 and TNFα was observed.

### Milk Whey Cytokine Responses

E. coli express an array virulence factors and immunogenic pathogen-associated molecular patterns (PAMPs) such as lipopolysaccharide (LPS) which contribute to the immunopathology in cases of coliform mastitis. The early immune response to E. coli intramammary infections is initiated by tissue resident cells including epithelial cells, macrophages, etc. which leads to the production of cytokines and chemokines (CXCL8, IL-1B, TNFa, IL-6, IL-10, etc.) which drive rapid recruitment and activation of leukocytes from the blood. As bacterial load increases the increase in PAMPs, especially LPS in the case of coliform mastitis, correlates with amplifications in cytokine production and leukocyte recruitment. The clinical outcome in coliform mastitis is predominantly driven by the host immune response. Clinical severity worsens and prolongs when the host is unable to efficiently clear the invading pathogen and/or the host is unable to regulate the inflammatory response leading to tissue damage. When infection is not controlled, the increase in bacterial load and PAMPs leads to further inflammatory responses which contribute to increases in clinical severity and pathology.

Here we demonstrate that CATH2 prevents acerbated inflammatory state in response to an experimental E. coli challenge while maintaining effective bacterial clearance. The investigators assessed cytokine levels in milk whey longitudinally over the course of E. coli infection comparing animals who received intramammary doses of saline to those who received CATH2.

One hundred fifty bovine milk samples were obtained in 3 separate cohorts and processed to whey. The whey samples were analyzed for total protein levels and the levels of 8 total cytokines using the developed MSD whey U-Plex method.

Animals that received CATH2 doses had a reduction in peak cytokine responses at 12 hr post-challenge in pro- and anti-inflammatory cytokines IL-1B, IL-6, IL-8/CXCL8, IL-10, and TNFα compared to animals which received saline controls. The reduction in cytokine response at 12 hr post-challenge may indicate treatment with CATH2 modulated host immune cells to either, promote rapid clearance of bacteria by tissue resident macrophages or enhance ability of tissue residents cells to rapidly recruit phagocytes to clear bacteria faster, and/or regulate the inflammatory cytokine responses to prevent immunopathology caused by the host response to E. coli. Based on in vitro mechanism work, both modes of action may be driven by CATH2.

In summary, CATH2 peptide treatment showed a significant effect in prevention of clinical mastitis, with 91.3% of animals never reaching the criteria for disease, with 87.0% prevention of intramammary infection entirely. Following administration of CATH2 there was a statistically significant initial increase in milk leukocytes in the treated quarters as compared to all other treatment groups but following challenge these animals were able to control SCC levels and return to baseline by 24 hours post challenge. Furthermore, at 12 hours post-challenge, animals in saline treated group reached peak temperature spike whereas CATH2 treated animals showed significant reductions in relevant proinflammatory cytokines and no temperature spike.

Based on these results, the inventors of the instant application have demonstrated that the CATH2 peptide administered by intramammary infusion effectively treats clinical mastitis.

### Example 2

### Treating Mastitis by CATH2 and its Analogs

The objective of this study was to evaluate the efficacy of Cathelicidin-2 peptide analogs for reduction of incidence of clinical mastitis and severity of infection following Escherichia coli intramammary (IMM) infusion in lactating dairy cows.

Healthy Holstein cows in their second or third lactation were selected based on culture negative results for mastitis pathogens and low somatic cell counts≤200,000. Enrolled animals were randomly assigned to treatment. Cows were commingled, housed and fed according to standard site procedures, and fitted with sensors to monitor activity. Cows were milked twice daily at regular intervals. The assigned treatment was administered following am and pm milkings starting 2 days prior to challenge. Four total treatments and the challenge were administered in the left front quarter. Treatment was administered twice daily for 2 days prior to challenge. The E. coli challenge material was administered to the treated quarter on the morning of Day 0 of study. The treatment regimen was designed to evaluate efficacy for prevention of the disease. All peptides were dosed at 50 mg/10 mL per dose. Milk samples were collected for analysis at 6-hour intervals on Day 0, and at each morning's milking from Day 1 through Day 7, and on Days 11 and 14.

TABLE 2
The amino acid sequences of CATH2 and its analog “Analog 2”

| | 1 | 2 | 3 | 4 | 5 | 6 | 7 | 8 | 9 | 10 | 11 | 12 | 13 | 14 | 15 | 16 | 17 | 18 | 19 | 20 | 21 |
|---|---|---|---|---|---|---|---|---|---|---|---|---|---|---|---|---|---|---|---|---|---|
| CATH2 | R | F | G | R | F | L | R | K | I | R | R | F | R | P | K | V | T | I | T | I | Q |
| (SEQ ID NO.: 35) | | | | | | | | | | | | | | | | | | | | | |
| Analog 2 | R | C | G | R | F | L | R | K | I | R | P | F | R | R | K | V | T | I | T | C | Q |
| (SEQ ID NO.: 36) | | | | | | | | | | | | | | | | | | | | | |

TABLE 3
The amino acid sequences of CATH2 and its analog “Analog 1”

| | 1 | 2 | 3 | 4 | 5 | 6 | 7 | 8 | 9 | 10 | 11 | 12 | 13 | 14 | 15 | 16 | 17 | 18 | 19 | 20 | 21 |
|---|---|---|---|---|---|---|---|---|---|---|---|---|---|---|---|---|---|---|---|---|---|
| CATH2 | R | F | G | R | F | L | R | K | I | R | R | F | R | P | K | V | T | I | T | I | Q |
| (SEQ ID NO.: 35) | | | | | | | | | | | | | | | | | | | | | |
| Analog 1 | R | F | G | R | W | L | R | K | I | R | R | Y | R | G | K | V | T | I | T | I | Q |
| (SEQ ID NO.: 37) | | | | | | | | | | | | | | | | | | | | | |

Clinical mastitis was observed in 100.0% of the negative control animals treated with the vehicle control, while only 47.06% of animals treated with CATH2 1-21L peptide met criteria for clinical mastitis, demonstrating a significant decrease in incidence of E. coli clinical mastitis in this model. The analogs Analog 1 and Analog 2 also showed an ability to prevent clinical mastitis at 78.95 and 95.74%, respectively, FIG. 9. Intramammary infection was observed in 100, 52.94, 94.74 and 94.74%, respectively.

As shown in FIG. 10, treatment with CATH2 1-21L peptide demonstrated the greatest reduction in the bacterial load post challenge. Each analog tested showed a reduction in bacterial load as compared to the vehicle control. The level of reduction correlates with the observed clinical scores for animals in each treatment group and incidence of clinical mastitis.

As shown in FIG. 11, there were no differences between the treatment groups for the percentage of animals ever having high somatic cell counts (SCC), although the vehicle-treated controls had a higher and later SCC peak compared to the treated groups. The vehicle-treated cows also had peak E. coli counts from the challenged quarter of >4.0 log₁₀ CFU/mL compared to <1.5 log₁₀ CFU/mL for CATH2 1-21L treated cows.

The CATH2 1-21L treated cows showed 41.48% with normal challenged quarter on Day 6 vs 0% for vehicle control treated animals. A return to normal milk is indicated by SCC <200,000, no isolation of E. coli, and no clinical scores for milk appearance or udder condition. The return to normal milk is an indication of reduced severity of disease with more rapid resolution of infection.

The analysis of fever showed that 80.0% of vehicle control treated (T01) cows demonstrated fever while only 41.2% of CATH2 (T02) treated cows showed increased temperature (See FIG. 12). The duration of fever was also significantly shorter for T02 cows compared to T01 cows (2.4 h for T02 vs 5.5 h for T01).

The bovine serum albumin (BSA) levels in milk are indicative of the level of inflammation and damage to the challenged quarter. As shown in FIG. 13, the CATH2 1-21L peptide treated animals show the lowest level of BSA in the milk as compared to the vehicle control, however, both analogs also show a reduction in BSA levels indicating a reduction in severity of disease.

The milk concentrations of BSA, milk haptoglobin, milk amyloid A, IL-1 beta, IL-6, IL-8, IL-10 and TNFα were highest in the vehicle-treated controls and lowest in the CATH2 1-21L treated cows (See FIGS. 13-20). Both analogs, Analog 1 and Analog 2, showed modulation on acute phase proteins and key cytokines demonstrating a relevant biological effect on infection, however to a lesser magnitude than that of CATH 1-21L.

Overall, the CATH2 1-21L peptide treatment provided the best protection against mastitis following IMM challenge with E. coli. Relatively limited protection against mastitis was provided by the other treatments evaluated in this study (Analog-1, 50 mg/10 mL and Analog-2, 50 mg/10 mL).

### Example 3

### Treating Mastitis by CATH2

S. aureus Challenge Study

### In Vivo Efficacy

Bovine mastitis specific bacterial pathogens were used to experimentally infect periparturient cows and first lactation heifer Holstein or Holstein Crossbred cattle to test the efficacy of the CATH2 peptide in the treatment and prevention of clinical mastitis.

A frozen stock of S. aureus was used to prepare a suspension of approximately 600 colony forming units in 5 mL. Standard plate counts were conducted on the challenge suspension immediately after preparation and after all animals were challenged. Details of each challenge inoculum preparation including concentration (CFU/5 mL) for pre- and post-challenge preparation were documented and confirmed to be in range.

Quarters that were bacteriologically positive for S. aureus were regarded as infected. Clinical mastitis was defined as a challenged quarter having a clinical score of at least 1 for either milk appearance or udder evaluation with an isolation of S. aureus (≥100 cfu/mL) from the quarter within 2 days of clinical signs. Sub-clinical chronic mastitis was defined as isolation of S. aureus (≥100 cfu/mL) from a challenged quarter and elevated SCC (>200,000 cells/mL) in the absence of abnormal milk or udder score.

All treatments were administered in accordance with the randomization provided by the biometrician into the challenged quarter (left front) of the udder. Treatments were administered upon onset of clinical mastitis. In this model, onset is approximately 1 to 3 days post-challenge. Upon confirmation of clinical mastitis, treatments were administered at the next milking. Treatments were administered with a 10 mL syringe via intramammary infusion and recorded.

Each animal with clinical mastitis received a total of 6 doses at 50 mg/10 mL by intramammary infusion administered twice daily for 3 consecutive days. Treatment with the CATH2 1-21L peptide resulted in 33.3% of animals achieving clinical cure. Animals also showed reduced severity of disease in this study as demonstrated by reduction in bacterial counts and reduced somatic cell counts. A total of 55.6% animals showed a positive response to treatment with CATH2 1-21L peptide.

As part of this first bovine study assessment on the drivers of efficacy, it was determined that CATH2 1-21L peptide was binding to milk protein in the milk upon administration by intramammary infusion. This highly charged molecule binds promiscuously to milk proteins, neutralizing the antibacterial activity.

The CATH2 peptide has antibacterial activity where it causes membrane disruption and lysis of the bacterial cell. The peptide also has cytotoxic effects on eukaryotic cells at high concentrations due the high positive charge, +8, and its ability to integrate in the cell membrane, resulting in membrane disruption. To further understand the behavior of the peptide in the context of the mammary gland, the inventors investigated the antibacterial activity and cytotoxicity of the peptide in the presence of milk. A modified microbiological assay to evaluate the minimal bactericidal concentration of the peptide in varying concentrations of milk and bacterial growth medium showed that the peptide antibacterial activity was completely inhibited in the presence of as little as 10% whole and skim milk. This finding led the inventors to investigate the impact of milk protein binding on peptide activity.

In a milk fraction bioanalysis assay for milk binding where the target milk concentration was 1000 ng/ml, CATH2 in 40 mL whole milk was collected from 2 separate healthy animals. The milk samples were subjected to a series of centrifugation steps to fractionate portions of milk to fat, whey, cell pellet, and protein pellet. Roughly 75% of the peptide was present in the protein pellet, which is assumed to be largely made up of casein. There was a small portion associated with whey, indicating that the peptide is promiscuously binding to protein in the milk.

In a follow up assay to better understand the impact of milk protein binding on antibacterial activity that may be seen in vivo, a ratio of peptide to milk protein was estimated. In this assay varying concentrations of milk in bacterial growth medium was incubated with 2 mg of peptide. Two minutes after adding peptide to the tube, 500 colony forming units (CFU)/mL of the E. coli challenge strain was added. The timing was purposeful for understanding how quickly the peptide can be completely bound. The low inoculum was also chosen intentionally to more closely replicate the dose provided in the in vivo challenge model, thereby providing a worst-case scenario on exposure to free peptide. The results showed that 30% whole milk was sufficient to completely block the antibacterial activity of the CATH2 peptide following a 2-minute incubation. The quantity of milk protein was enough to bind 2 mg of CATH2 peptide. The bacterial growth was evaluated at 1, 2, 4, 6, and 24 hours post inoculation. Two milligrams of CATH2 in bacterial medium alone was completely bactericidal with E. coli below the level of detection at all time points. While some inhibition of bacterial growth was seen at 10% and 20% milk, the relative antibacterial activity was greatly reduced as compared to media alone. The lower level of detection was 10 CFU/mL and the upper level of quantification measured for the purposes of this assay was set to 1.5×105 CFU/mL.

### Example 4

### Treating Mastitis by CATH2

S. uberis Challenge Study

A contemporary Streptococcus uberis isolate was identified in the culture collection showing robust adherence and invasion capacity in in vitro cell culture with primary mammary epithelial cells. This isolate was chosen for development of a S. uberis bovine challenge model. The resulting model represented a severe S. uberis clinical mastitis with a highly virulent strain. In many animals the challenge isolate would spread to unchallenged quarters resulting in severe disease. Animals were removed from study upon reaching predetermined criteria for humane removal from study.

The culture was stored at approximately −70° C. in Todd Hewitt broth (TSB)-10% glycerol. Before challenge, an aliquot from the frozen vial was used to inoculate a blood agar plate. After overnight incubation at 37° C., a 1 μL loopful of bacteria was used to inoculate fresh Todd Hewitt broth for further incubation for 7 hours at 37° C. The stock solution was diluted 10-fold to achieve a suspension of ˜5.0×10³ CFU/mL. This material was then diluted in PBS to achieve the final concentration of 5×10² CFU/5 mL dose.

A total of 69 animals, 23 animals per treatment group, were included on study. The experimental design of the study was a completely randomized design with a one-way treatment structure replicated in two batches. The first batch included 30 animals. The second batch included 39 animals. Each treatment group was represented in each batch of the study. Animals received the challenge material in a 5-mL dose by intramammary infusion in the left front quarter on Day 0 after PM milking. At the designated time points per the study design relative to challenge animals received a saline or drug dose in the left front quarter.

TABLE 4
S. uberis Challenge Experiments

| | Number of | Challenge | Treatment | | Route of |
|---|---|---|---|---|---|
| Treatment | Animals | Administration | Description/Dose | Dosage | Administration |
| T01 | 23 | Streptococcus | Saline | 4 doses, at −12, | Intramammary |
| | | uberis | | 0, 12, and 24 | Infusion |
| | | Day 0 | | hours | |
| | | | | respective of | |
| | | | | challenge | |
| T02 | 23 | | CATH2 in | 4 doses, at −12, | |
| | | | aqueous | 0, 12, and 24 | |
| | | | formulation; | hours | |
| | | | 50 mg/10 mL | respective of | |
| | | | | challenge | |
| T03 | 23 | | CATH2 in | 2 doses, −48 | |
| | | | aqueous | hours and −24 | |
| | | | formulation; | hours | |
| | | | 50 mg/10 mL | | |

−12, 0, 12, and 24 hours refer to the hours of saline or treatment administration relative to S. uberis challenge administration.

Animals were observed for 12 days post challenge. Animals were removed from study per predetermined criteria set in the protocol for humane removal from study. In the Kaplan Meier Analysis, FIG. 21, CATH2 treatment either with 2 doses prior to challenge or 4 doses spanning the time of challenge, demonstrates a reduction in the number of animals meeting criteria for removal of study indicating a reduction in the severity of infection in this challenge model system.

In addition to a reduction in severity of disease, CATH2 treatment also resulted in reduced incidence of clinical mastitis. The 4 dose regimen demonstrated a greater capacity to inhibit the progression of clinical mastitis. While both treatment regimens resulted in similar reduction in severity of disease.

In summary, based on these results, the inventors of the instant application have demonstrated that the CATH2 peptide effectively treats clinical mastitis by driving modulation of the immunoresponse.

### Example 5

### Evaluation of CATH2 for Dry Off Therapy in Dairy Cattle

In an additional study, pregnant multiparous Holstein cattle were treated with a single dose of CATH2 at approximately 60 days prior to expected calving dates for this group of animals by intramammary infusion following milking. These animals were considered to be entering the so-called dry period in which animals are no longer milked until post-partition. The objective of this study was to understand the immunomodulatory activity of varying doses of CATH2 administered by intramammary infusion in healthy multiparous cows at dry off.

TABLE 5
Evaluation of CATH2 for Dry Off Therapy in Dairy Cattle

| | Number of | Treatment Description/ | | Route of |
|---|---|---|---|---|
| Treatment | Animals | Dose for LR quarter | Dosage for LR quarter | Administration |
| T01 | 6 | CATH2 - | 100 mg in 10 mL Dose Day 0 - | Intramammary |
| | | 100 mg/10 mL | at Dry Off - Left Rear | Infusion |
| | | | quarter | |
| T02 | 5 | CATH2 - | 200 mg in 10 mL Dose Day 0 - | |
| | | 200 mg/10 mL | at Dry Off - Left Rear | |
| | | | quarter | |
| T03 | 5 | CATH2 - | 400 mg in 10 mL Dose Day 0 - | |
| | | 400 mg/10 mL | at Dry Off - Left Rear | |
| | | | quarter | |

Udder secretion samples were collected 1, 2, and 7 days post administration. Samples were also collected from the neighboring Right Rear quarter as an untreated in animal control of varying immunological responses. The samples were processed for transcriptomics analysis to determine the healthy animal response to treatment alone.

Consistent with results observed in the E. coli lactating cow model system, CATH2 demonstrates a treatment effect in healthy dry cows resulting in shifts in gene expression levels of similar gene sets in both dry cow and lactating cow systems. Prior to challenge in the lactating cow model, there is a consistent treatment effect of CATH2 alone indicating a modulation of the animal immune response. In the lactating cow model, these shifts lead to an enhanced response in clearance of the E. coli and resolution of inflammation as compared to untreated controls. In observing similar effects in healthy dry cows, these data indicate that animals treated at dry off will also have an enhanced ability to clear invading bacteria during the dry period.

Key examples of similar gene expression profiles include, but are not limited to, increased expression of: CCL24, CXCL2, IL1RAP, CXCR1, and CXCR2. These observations were seen at the 1-day time point post administration of 400 mg/10 mL dose level in the healthy dry cows as compared to 12 hour time point post administration of 100 mg/10 mL dose level in the lactating cows.

Having described preferred embodiments of the invention, it is to be understood that the invention is not limited to the precise embodiments, and that various changes and modifications may be effected therein by those skilled in the art without departing from the scope or spirit of the invention as defined in the appended claims.

1-50. (canceled)

51. A method for treating a mastitis in a subject, the method comprising:
administering to said subject cathelicidin 2 (CATH2) peptide, wherein said peptide comprises the amino acid sequence set forth in SEQ ID NO.: 35, 36, or 37,
thereby treating said mastitis in said subject.

52. The method of claim 1, wherein said mastitis is associated with or induced by a bacteria.

53. The method of claim 52, wherein said bacteria is E. coli.

54. The method of claim 52, wherein said bacteria is S. aureus.

55. The method of claim 52, wherein said bacteria is S. uberis.

56. The method of claim 51, wherein said peptide is capable of being effective in a mammary gland.

57. The method of claim 51, wherein said peptide is an immune modulatory peptide.

58. The method of claim 51, wherein the effectiveness of said peptide is induced by immuno-modulation.

59. The method of claim 51, wherein said peptide has no antibiotic activity.

60. The method of claim 51, wherein said peptide has no direct killing effect on said bacteria.

61. The method of claim 51, wherein said administration is an intra-mammary administration.

62. The method of claim 51, wherein said subject is a bovine.

63. The method of claim 51, wherein said subject is a dairy cow.

64. The method of claim 51, wherein the treatment is an immuno-modulatory treatment.


SEQUENCE LISTING (37 sequences)

SEQ ID NO: 1 - protein, 27 aa, synthetic construct
  REGION 1..27: note = CMAP27
RFGRFLRKIRRFRPKVTITIQGSARFG

SEQ ID NO: 2 - protein, 21 aa, synthetic construct
  REGION 1..21: note = cycCMAP(1-21)[Lys8]
  SITE 8: note = MISC_FEATURE - is Fmoc-L-bishomopropargylglycine
  SITE 21: note = MISC_FEATURE - azido resin attached
RFGRFLRXIRRFRPKVTITIQ

SEQ ID NO: 3 - protein, 21 aa, synthetic construct
  REGION 1..21: note = cycCMAP(1-21)[Arg7]
  SITE 7: note = MISC_FEATURE - is Fmoc-L-bishomopropargylglycine
  SITE 21: note = MISC_FEATURE - azido resin attached
RFGRFLXKIRRFRPKVTITIQ

SEQ ID NO: 4 - protein, 21 aa, synthetic construct
  REGION 1..21: note = cycCMAP(1-21)[Leu6]
  SITE 6: note = MISC_FEATURE - is Fmoc-L-bishomopropargylglycine
  SITE 21: note = MISC_FEATURE - azido resin attached
RFGRFXRKIRRFRPKVTITIQ

SEQ ID NO: 5 - protein, 21 aa, synthetic construct
  REGION 1..21: note = cycCMAP(1-21)[Leu6],Phe2/Trp
  SITE 6: note = MISC_FEATURE - is Fmoc-L-bishomopropargylglycine
  SITE 21: note = MISC_FEATURE - azido resin attached
RWGRFXRKIRRFRPKVTITIQ

SEQ ID NO: 6 - protein, 21 aa, synthetic construct
  REGION 1..21: note = cycCMAP(1-21)[Leu6],Phe2,5/Trp
  SITE 6: note = MISC_FEATURE - is Fmoc-L-bishomopropargylglycine
  SITE 21: note = MISC_FEATURE - azido resin attached
RWGRWXRKIRRFRPKVTITIQ

SEQ ID NO: 7 - protein, 21 aa, synthetic construct
  REGION 1..21: note = cycCMAP(1-21)[Leu6],Phe2,5,12/Trp
  SITE 6: note = MISC_FEATURE - is Fmoc-L-bishomopropargylglycine
  SITE 21: note = MISC_FEATURE - azido resin attached
RWGRWXRKIRRWRPKVTITIQ

SEQ ID NO: 8 - protein, 21 aa, synthetic construct
  REGION 1..21: note = cycCMAP(1-21)[Leu6],Phe5,12/Trp
  SITE 6: note = MISC_FEATURE - is Fmoc-L-bishomopropargylglycine
  SITE 21: note = MISC_FEATURE - azido resin attached
RFGRWXRKIRRWRPKVTITIQ

SEQ ID NO: 9 - protein, 21 aa, synthetic construct
  REGION 1..21: note = cycCMAP(1-21)[Leu6],Phe12/Trp
  SITE 6: note = MISC_FEATURE - is Fmoc-L-bishomopropargylglycine
  SITE 21: note = MISC_FEATURE - azido resin attached
RFGRFXRKIRRWRPKVTITIQ

SEQ ID NO: 10 - protein, 27 aa, synthetic construct
  REGION 1..27: note = Synthetic Peptide
GFRASGQITITVKPRFRRIKRLFRGFR

SEQ ID NO: 11 - protein, 21 aa, synthetic construct
  REGION 1..21: note = Synthetic Peptide
QITITVKPRFRRIKRLFRGFR

SEQ ID NO: 12 - protein, 18 aa, synthetic construct
  REGION 1..18: note = Synthetic Peptide
QITITVKPRFRRIKRLFR

SEQ ID NO: 13 - protein, 15 aa, synthetic construct
  REGION 1..15: note = Synthetic Peptide
QITITVKPRFRRIKR

SEQ ID NO: 14 - protein, 15 aa, synthetic construct
  REGION 1..15: note = Synthetic Peptide
QITITVKPRWRRIKR

SEQ ID NO: 15 - protein, 15 aa, synthetic construct
  REGION 1..15: note = Synthetic Peptide
QITITVKPRYRRIKR

SEQ ID NO: 16 - protein, 12 aa, synthetic construct
  REGION 1..12: note = Synthetic Peptide
QITITVKPRWRR

SEQ ID NO: 17 - protein, 26 aa, synthetic construct
  REGION 1..26: note = Synthetic Peptide
RFGRFLRKIRRFRPKVTITIQGSARF

SEQ ID NO: 18 - protein, 21 aa, synthetic construct
  REGION 1..21: note = Synthetic Peptide
RFGRFLRKIRRFRPKVTITIQ

SEQ ID NO: 19 - protein, 18 aa, synthetic construct
  REGION 1..18: note = Synthetic Peptide
RFLRKIRRFRPKVTITIQ

SEQ ID NO: 20 - protein, 15 aa, synthetic construct
  REGION 1..15: note = Synthetic Peptide
RKIRRFRPKVTITIQ

SEQ ID NO: 21 - protein, 15 aa, synthetic construct
  REGION 1..15: note = Synthetic Peptide
RKIRRWRPKVTITIQ

SEQ ID NO: 22 - protein, 15 aa, synthetic construct
  REGION 1..15: note = Synthetic Peptide
RKIRRYRPKVTITIQ

SEQ ID NO: 23 - protein, 12 aa, synthetic construct
  REGION 1..12: note = Synthetic Peptide
RRWRPKVTITIQ

SEQ ID NO: 24 - protein, 15 aa, synthetic construct
  REGION 1..15: note = Synthetic Peptide
RFGRFLRKIRRFRPK

SEQ ID NO: 25 - protein, 21 aa, synthetic construct
  REGION 1..21: note = Synthetic Peptide
RCGRFLRKIRPFRRKVTITRQ

SEQ ID NO: 26 - protein, 21 aa, synthetic construct
  REGION 1..21: note = Synthetic Peptide
RCGRFLRKIRPFRGKVTITRQ

SEQ ID NO: 27 - protein, 21 aa, synthetic construct
  REGION 1..21: note = Synthetic Peptide
RFGRFLRKIRRFRGKVTITRQ

SEQ ID NO: 28 - protein, 21 aa, synthetic construct
  REGION 1..21: note = Synthetic Peptide
RWGRWLRKIRRWRPKVTITRQ

SEQ ID NO: 29 - protein, 21 aa, synthetic construct
  REGION 1..21: note = Synthetic Peptide
RWGRWLRKIRRWRPKVTITIQ

SEQ ID NO: 30 - protein, 18 aa, synthetic construct
  REGION 1..18: note = Synthetic Peptide
RFLRKIRRFRPKVTITRQ

SEQ ID NO: 31 - protein, 18 aa, synthetic construct
  REGION 1..18: note = Synthetic Peptide
RFLRKIRRFRGKVTITRQ

SEQ ID NO: 32 - protein, 18 aa, synthetic construct
  REGION 1..18: note = Synthetic Peptide
RWLRKIRRWRPKVTITIQ

SEQ ID NO: 33 - protein, 18 aa, synthetic construct
  REGION 1..18: note = Synthetic Peptide
RWLRKIRRWRPKVTITRQ

SEQ ID NO: 34 - protein, 18 aa, synthetic construct
  REGION 1..18: note = Synthetic Peptide
RWLRKIRRWRGKVTITRQ

SEQ ID NO: 35 - protein, 21 aa, synthetic construct
  REGION 1..21: note = Synthetic Peptide
RFGRFLRKIRRFRPKVTITIQ

SEQ ID NO: 36 - protein, 21 aa, synthetic construct
  REGION 1..21: note = Synthetic Peptide
RCGRFLRKIRPFRRKVTITCQ

SEQ ID NO: 37 - protein, 21 aa, synthetic construct
  REGION 1..21: note = Synthetic Peptide
RFGRWLRKIRRYRGKVTITIQ